(19) United States
(12) Patent Application Publication
(10) Pub. No.: US 2017/0136091 A1
(43) Pub. Date: May 18, 2017

(54) FOOD CONTAINING PROLACTIN

(71) Applicant: Technion Research & Development Foundation Ltd., Haifa (IL)

(72) Inventor: Naim SHEHADEH, Haifa (IL)

(21) Appl. No.: 15/420,469

(22) Filed: Jan. 31, 2017

Related U.S. Application Data
(63) Continuation of application No. 14/625,358, filed on Feb. 18, 2015, now abandoned.
Application No. 14/625,358 is a continuation of application No. 13/201,941, filed on Oct. 31, 2011, now abandoned.
(60) Provisional application No. 61/153,080, filed on Feb. 17, 2009.

(51) Int. Cl. A61K 38/22; A23J 3/08; A23P 10/30; A23L 2/66; A23L 33/17; A23L 33/00; A61K 9/16; A23K 20/147

(52) U.S. Cl. CPC A61K 38/2257; A61K 9/1676; A61K 9/1652; A61K 9/1694; A23J 3/08; A23K 20/147; A23L 2/66; A23L 33/17; A23L 33/40; A23P 10/30; A23V 2002/00

(57) ABSTRACT

A nutritional composition for a subject, comprising prolactin identical or similar or analogous to prolactin found in a natural food source, and at least one protective layer, wherein release of said prolactin from the composition in said subject is the result of an environmental event.

### CROSS REFERENCE

This application is a continuation of U.S. patent application Ser. No. 14/625,358 filed Feb. 18, 2015, which is a continuation of U.S. patent application Ser. No. 13/201,941 filed Oct. 31, 2011, which is a US National Phase filing of PCT/IL2010/000139 filed Feb. 17, 2010, which claims priority to U.S. Provisional Patent Application Ser. No. 61/153,080 filed Feb. 17, 2009, each incorporated by reference herein in its entirety.

### FIELD OF THE INVENTION

This invention relates to prolactin, its incorporation and delivery in nutritional food and feed and its application in supplementing the diet of mammals.

### BACKGROUND OF THE INVENTION

One of the major effects of breast-feeding is the support of gut maturation. Breast-fed milk may also influence small intestinal growth and functional maturation.

Prolactin has been detected in milk of the cow (Erb et al., 1977), goat (McMurty and Malven, 1974), Pig (Mulloy and Malven, 1979) and primates (Gala et al., 1975).

Prolactin is a polypeptide hormone that is now appreciated to have over 300 separate biological activities, including promoting lactation in mammals, and roles in reproduction and homeostasis (Freeman et al., 2000).

Prolactin may also be an important agent in the development of neuroendocrine, reproductive and immune function in neonatal mice and rats (Sinha and Vanderlaan, 1982, Grove et al., 1991). Moreover, prolactin in human milk may have a physiological significance for newborn humans, including enhancing calcium absorption across intestinal epithelial membranes (Taketani and Mizuno, 1985, Amnattananakul et al., 2005).

Prolactin receptors (long and short forms) were identified in the epithelial cells of rat duodenum, jejunum, ileum and colon (Ouhtit et al., 1994). In humans, duodenal enterocytes also express prolactin receptors and prolactin receptors are expressed in villous columnar epithelial cells of the duodenum from 12 to 14 weeks of gestation (Freemark et al., 1997). Indeed, prolactin has been found to be absorbed in the intestine of neonates (Amnattananakul et al., 2005). Therefore, the small intestine in newborn humans may be a direct target organ of prolactin.

However, human neonates and newborn animal infants are frequently weaned of their natural food or feed immediately or shortly after birth, and are then nourished primarily with artificially produced food substitutes.

A putative alternative exogenous source of prolactin is unprocessed milk and eggs. However, most industrial food or feed production processes involve manufacturing conditions that are destructive to the viability of prolactin. In addition, supply chain constraints impose longer shelf life requirements such that extended storage under adverse conditions is enabled. Unfortunately, such requirements often result in loss of efficacy of the biological activity of such compound. Indeed, we have shown that prolactin concentration is significantly higher in fresh human milk (329.5±268.5 mU/L) compared with pasteurized human milk (120.8±103 mU/L). In addition, only trace amounts of prolactin were detected in commercial bovine fresh milk and in infant formulas (Vaxman and Shehadeh, 2008).

Food components destroyed during processing of food may be extracted from plants, recombinant organisms or otherwise artificially generated, and may be produced at a lower cost, be free of bacteria and viruses frequently found in traditional sources, and be better accepted as healthier and safer by both regulatory authorities and the general public. The technology for production of human DNA recombinant prolactin is available (Price et al., 1995).

Although a source of prolactin for newborn babies is mother's milk, and despite evidence indicating that this source of prolactin may have a physiological importance (Taketani and Mizuno, 1985), it is nevertheless at present not clear whether prolactin should be added to food preparations for babies. For example, in suckling rats only 16% of the total plasma prolactin originates from mother's milk (Gonella et al., 1989; Whitworth and Grosvenor, 1995). In addition, angiogenesis, the development of blood vessels, has been found to be inhibited by proteolytic fragments of native prolactin (Clapp C and De La Escalera G M, Prolactins: novel regulators of angiogenesis, News Physiol Sci 12: 231-237, 1997).

A further factor complicating the decision of whether, and also how, to add prolactin to food stuffs is that there are many variants of prolactin (Freeman, 2000), the disclosures in their entirety hereby expressly incorporated by reference herein for the purpose of indicating the background of embodiments of the invention and illustrating the state of the art.

Due to the great variance, bioassays and immunoassays can give different results due to differing glycolyzation, phosphorylation sulfation, and deamidation and degradation extents. The prolactin molecules may result from alternative splicing of the primary transcript, proteolytic cleavage and other post-translational modifications of the amino acid chain, and prolactin molecules may interact between themselves to produce dimer and polymer prolactin or with other proteins such as immunoglobulins. The roles of these variants are unclear.

Therefore, there is at present uncertainty as to which form, if any, of prolactin should be provided in baby foods, and how to prevent its degradation during preparation and storage.

Despite reservations regarding adding prolactin to food, we have recognized a need for the addition and have devised a nutritional feed composition that will answer the need for optimal nutrition to newborn humans, capable of delivering biomaterials including prolactin in a manner that will guarantee its viability to the infant, as well as along the supply chain of its manufacture and similar need is recognized in supplementing the nutritional formula of term and preterm human neonates.

### SUMMARY OF THE INVENTION

A nutritional composition for a subject is provided, the composition comprising prolactin identical or similar or analogous to prolactin found in a natural food source, and at least one protective layer, wherein release of said prolactin from the composition in said subject is the result of an environmental event.

A method is provided for enhancing small intestinal growth, small intestinal functional maturation and/or calcium absorption across intestinal epithelial membranes enhancing gut maturation, and/or stimulating mitosis in T lymphocytes, and/or increasing rate of weight gain, and/or improving the FCR, and/or reducing incidence of diarrhea and/or other gastric disorders and/or increasing the life expectancy of a subject, the method comprising:

A method for preparing protected prolactin in a nutritional food or feed or drink is provided, the method comprising:
preparing a protected prolactin comprising: mixing said prolactin with a first protective material forming a first blend;
processing the first blend to form a dry second blend comprising at least one protective layer,
wherein said protective layers are specifically designed to degrade as a response to change in an environmental trigger, and adding the protected prolactin to said nutritional food or nutritional feed or drink.

A supplemented nutritional food or feed or drink of a mammal, may comprise any one of said compositions, incorporated in said nutritional food or feed or drink, the composition thereby being a supplement to said food or feed or drink.

The supplemented nutritional drink may have a level of prolactin between 40 and 800 mU/L. Preferably, the supplemented nutritional drink has a level of prolactin between 100 and 600 mU/L.

In the supplemented nutritional food the food may be selected from baby or newborn or infant formulas, dried milk, and milk substitute.

In the supplemented nutritional food the formula may be a dry powder and the prolactin composition may be dry.

Alternatively, the baby formula is a liquid prepared from a dry powder.

Preferably, the prolactin composition is evenly distributed in the food.

Prolactin may also be provided for manufacture of a nutritional composition for enhancing mall intestinal growth, small intestinal functional maturation and/or calcium absorption across intestinal epithelial membranes enhancing gut maturation, and/or stimulating mitosis in T lymphocytes, and/or increasing rate of weight gain, and/or improving the FCR, and/or reducing incidence of diarrhea and/or other gastric disorders and/or increasing the life expectancy of a subject, the composition comprising prolactin identical or similar or analogous to prolactin found in a natural food source, and at least one protective layer, wherein release of said prolactin from the composition in said subject is the result of an environmental event.

Prolactin may also be provided for manufacture of a supplemented nutritional food or feed or drink for enhancing small intestinal growth, small intestinal functional maturation and/or calcium absorption across intestinal epithelial membranes enhancing gut maturation, and/or stimulating mitosis in T lymphocytes, and/or increasing rate of weight gain, and/or improving the FCR, and/or reducing incidence of diarrhea and/or other gastric disorders and/or increasing the life expectancy of a subject, the supplemented nutritional food comprising nutritional food and a prolactin composition, the prolactin composition comprising prolactin identical or similar or analogous to prolactin found in a natural food source, and at least one protective layer, wherein release of said prolactin from the composition in said subject is the result of an environmental event.
The supplemented nutritional food or feed or drink manufacture may comprise adding said prolactin composition to said nutritional food or feed or drink

The manufacture may comprise:
preparing a protected prolactin comprising:
mixing said prolactin with an appropriate first protective material forming a first blend;
processing the first blend to form a dry second blend comprising prolactin in at least one protective layer, wherein said protective layer is specifically designed to degrade as a response to change in an environmental trigger, and
adding the dry second blend to said nutritional food or nutritional feed or drink.

The preparing a protected prolactin may further comprise:
forming a round core from the second blend;
drying the core;
collecting the dehydrated core;
making a first suspension, comprising the dehydrated core in a liquid second protective material;
drying the first suspension in a fluidized bed drier;
collecting the fluidized bed-dried suspension;
making a second suspension, comprising the collected dried suspension in a liquid third protective material, and drying the second suspension in a fluidized bed drier.

Said first blend may be liquid.

Said first suspension may further comprise: a Maltodextrin, a vitamin, an antioxidant, a protease inhibitor, a growth hormone, an EGF (Epidermal Growth Factor), an insulin and insulin-like growth factor, an insulin-like growth factor's binding protein, an immunoglobulin, a proline-rich polypeptide, a lactoferrin, a protease, a lactalbumin, an interleukin, a lysozyme, a TGFA (Transforming Growth Factor A), a PDGF (Platelet Derived Growth Factor) or combination thereof.

Second suspension may further comprise: a maltodextrin, a vitamin, an antioxidant, a protease inhibitor, a growth hormone, an EGF (Epidermal Growth Factor), an insulin and insulin-like growth factor, an insulin-like growth factor's binding protein, an immunoglobulins, a proline-rich polypeptide, a lactoferrin, a protease, a lactalbumin, an interleukin, a lysozyme, a TGFA (Transforming Growth Factor A), a PDGF (Platelet Derived Growth Factor) or combination thereof.

In the supplemented nutritional food or feed or drink manufacture, maltodextrin may have a dextrose equivalent (DE) between 2 and 64, preferably 18.

Prolactin may be derivatized ex-vivo. Said derivatization may be done by enzymatic digestion, physical methods, chemical methods, or any combination thereof.

The supplemented nutritional food or feed or drink manufacture may further comprise an agglomeration step.

Said agglomeration step may result in particle average diameter between about 0.1 and about 5,000 micrometers.

The core may be inert.

Forming the round core may further comprise:
flash freezing said liquid blend;
collecting the droplets produced;
lyophilizing the collected droplets;
collecting the lyophilized droplets, thereby creating a round core. Said core may include prolactin.

Said third protective material may be designed to thermally protect prolactin for no less than 2 minutes at a temperature of no less than 95° C.

Said second protective material may be designed to protect said bioactive compound from proteolytic enzymes and pH of no more than 4.75.

### DETAILED DESCRIPTION OF THE INVENTION

Suckling in humans and other mammals has multiple beneficial effects on infants' well being, including optimal nutrition, protection against a wide range of infection related diseases and promoting small intestine growth and development.

In suckling rats only 16% of the total plasma prolactin originates from mother's milk (Gonella et al., 1989; Whitworth and Grosvenor, 1995). Nevertheless, we have realized an importance in supplementing the prolactin self-produced by babies with prolactin from exogenous sources such as formulas for preparation for bottle-feeding.

Accordingly, a nutritional composition for a subject is provided, the composition comprising microencapsulated prolactin.

Encapsulations that are considered suitable for such nutritional compositions have been described in full in WO05/115473, the disclosure in its entirety hereby expressly incorporated by reference herein for the purpose of indicating the background of embodiments of the invention and illustrating the state of the art.

The term “prolactins” includes fragments and/or derivatives of prolactin and/or compounds that are similar or analogous to prolactin found in a natural food sources such as natural unprocessed milk, unprocessed eggs, plant material, animal tissue, recombinant DNA technology, or the result of PCR, or the result of chemical synthesis.

The terms “analogous” or “analog” or “analogue” interchangeably refer to a structure that is similar in function to one in another kind of organism but is of dissimilar evolutionary origin.

According to one aspect, a method is provided to utilize prolactin which is analogous to milk and eggs prolactin or prolactin variants, as supplements for human infant foods and animal infant feeds.

In some embodiments, the mammal is a preterm human infant, term human infant, a human baby, human toddler, human adolescent, human adult or human old person.

In some embodiments, the mammal is a grown or mature animal.

In some embodiments, prolactin is supplemented to food, feed or drink at levels measured in human mother's milk, i.e. between about 40 and 800 mU/L; alternatively, prolactin may be supplemented to promote health or growth in infants. Levels of prolactin in the latter preparations for human infants are preferably at levels of 100 to 600 mU/L.

Preferably, the prolactin variants in the nutritional composition are either similar, or identical, to the variants present in natural unprocessed colostrum, a full milk or egg. Most preferably, variants are selected that are specifically suitable for specific mammals. In particular, preferably the prolactin variants in a nutritional composition for feeding a certain animal are similar or identical to those in the milk or colostrum of the same animal.

Optionally, all of the variants present in the colostrum, full milk or egg are included in the nutritional composition. Alternatively, the prolactin variants in the nutritional compositions are essentially non-glycosylated and/or non-phosphorylated and/or non-dimerized or polymerized.

In one embodiment, the ratio between of the variants present in natural unprocessed colostrum, full milk or egg is approximately the ratio of the same variants included in the nutritional composition. In an alternative embodiment the proportion of non-phosphorylated and non-glycosylated prolactin is higher than the proportion of these variants in unprocessed colostrum, full milk or egg.

In one embodiment, the release of the prolactin into the nutritional composition of the invention, or in another embodiment, directly to the subject receiving the nutritional compositions of the invention, is following exposure to an environmental trigger.

The term “trigger” refers to a change in environmental conditions sufficient to initiate degradation in the encapsulating materials of the encapsulating layers used in the composition and methods of the invention, the change leading to release of the bioactive, viable compounds encapsulated therein. In one embodiment, the reference environmental condition is time, or in another embodiment temperature, or in another embodiment moisture content, or in another embodiment pressure, or in another embodiment pH, or in another embodiment ionic strength, or in another embodiment enzymatic activity, or in another embodiment a combination thereof.

In one embodiment the environmental condition change may be selected as either a change of over ±2.5% in the reference environmental condition, or over ±5%, ±10, ±15%, ±20%, ±25%, ±30%, ±35%, ±40%, ±45% or ±50%.

In one embodiment, a protective layer surrounding or incorporating a prolactin is specifically designed to degrade, or in another embodiment, undergo controlled release, as a response to exposure to the change in environmental condition, which is in another embodiment time, or in another embodiment temperature, or in another embodiment moisture content, or in another embodiment pressure, or in another embodiment pH, or in another embodiment ionic strength, or in another embodiment enzymatic activity, or in another embodiment a combination thereof.

Therefore, in one embodiment, a core wherein prolactin is embedded, is coated with a protective material to protect the active core from digestion in a digestive system of a subject, and release the core which in another embodiment, releases the active compound, only as a response to an increase in pH.

In another embodiment, the active core, which is encapsulated in an encapsulating material allowing release of the core based on increase in pH, is further encapsulated with another encapsulating material, designed to protect the core from increased temperature as described herein.

The skilled artisan in the art, would recognize that the order of environmental triggers releasing prolactin is not rigid and depending on the environmental conditions of manufacturing, environmental conditions of integration into food or feed products, environmental conditions of storage after integration onto food or feed products, desired delivery location within the gastrointestinal system, timing and physiological activity desired, the encapsulating layers could accommodate those requirements without departing from the scope of the invention as described herein.

In one embodiment, any factor, which may affect the entrapment of the subject prolactin in a biodegradable matrix, and thereby affect its initial loading, in one embodiment, or, in another embodiment, subsequent release, or in another embodiment, a combination thereof, may be utilized according to the methods and compositions of this invention. In other embodiments, such factors may comprise inter-alia, the initial solvent concentration, its molecular size and polarity, the temperature and pressure under which the solvent is removed, molecular weight number (MWn) average of the biodegradable matrix, its polydispersity index, the size and polarity of the prolactin, when the biodegradable matrix is in another embodiment a polymer, the monomer ratio and distribution along the copolymer's chain, or a combination thereof.

In addition, D/L ratio within each monomer of a biodegradable polymer will affect release rates. In one embodiment, the term D/L ratio refers to the ratio of monomer molecules that affect the direction (D-right, L-left), in which a cross-polarized lens will be rotated when observing a single optically active monomer, like lactic acid. Since most mammals have D-specific enzymes, that ratio will affect the digestion rate of the biodegradable biopolymer, affecting its molecular weight and consequently its viscosity, thereby affecting release rate of any entrapped prolactin.

In one embodiment, complexes between the prolactin and the protective layer may be formed via covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In one embodiment, complexation may affect the compound's solubility in aqueous solution, eliminate aggregation, enhance the physical and/or chemical stability of the compound, change the immunogenicity or reactivity of the compound, promote gut maturation, small intestinal growth and/or functional maturation, enhance calcium absorption across intestinal epithelial membranes, stimulate mitosis in T lymphocytes or combination thereof.

The compositions may also serve to increase the rate of weight gain, improve the FCR (Feed Conversion Ratio) of newborn animals, reduce the mortality rate of newborn animals, prevent diarrhea or other gastric disorders or increase the life expectancy of newborn animals after birth.

In one embodiment, the invention provides a method for preparing an encapsulated prolactin in a nutritional food or feed, comprising mixing the prolactin with an appropriate encapsulating material in liquid form forming a blend, then processing the blend formed to form a functionally multilayered protected dry blend, wherein the protective layer is specifically designed in another embodiment, to degrade as a response to change in an environmental trigger, and then adding the dry blend to the nutritional food or feed, thereby preparing a multilayered encapsulation of the prolactin in a nutritional feed.

In another embodiment of the invention, a method is provided for the encapsulation of a prolactin, comprising; (i) mixing a prolactin with a wall-forming encapsulating material, and (ii) rapidly cooling the wall forming material thereby resulting in encapsulation of the prolactin.

In one embodiment, the abovementioned process produces a core of a matrix entrapping the prolactin, whereas in another embodiment, the core does not initially contain a prolactin and is therefore inert. In one embodiment, the core produced is substantially rounded, to improve the addition of additional encapsulating layers.

In one embodiment forming the round core further comprises flash freezing said liquid blend, collecting the droplets produced, lyophilizing the droplets collected and collecting the lyophilized droplets, thereby creating a round core, wherein said core may comprise a prolactin.

In one embodiment, encapsulation refers to the process where one or more prolactin, prolactin variant and/or prolactin analog are coated with, or in another embodiment, entrapped within, another food grade or feed grade or pharmaceutical grade material or matrix. Encapsulation of heat sensitive compounds, such as for example nutraceutical components, enzymes or bioactive proteins, into matrixes that are edible and digestible, is generally difficult for a number of reasons. Conventional encapsulation processes, which expose matrix material and encapsulants to high temperatures and moisture such as those encountered in pelleting and extrusion, causes thermal destruction or loss of biological viability of the encapsulant. Thus, either large initial load of encapsulant, a very expensive and potentially hazardous preposition, would be required, or the encapsulant would not stand the encapsulation process at all. If the encapsulant can be encapsulated into a matrix under sufficiently low temperatures, the resulting product is a solid that is characterized as a hard glass-like solid that is capable of being processed further to yield a flowable powder, amenable to additional processing.

In another embodiment, the temperature at which the particles are consumed, or in another embodiment, the eating temperature, is generally lower than 50 degrees Celsius, which is far below the glass transition temperature, Tg. Careful design of the glassy matrix can release the prolactin under desired conditions of temperature, moisture, pH or enzymatic environment.

The encapsulated matrix could be used in one embodiment as dense pellets for a variety of processing applications, where a controlled release of the heat sensitive encapsulant is desired. The physical hardness of the products and their mechanical stability are advantageous in one embodiment for many processing applications.

In one embodiment, the encapsulant is food grade, or in another embodiment, feed grade. In one embodiment, the encapsulant is a polysaccharide, or in another embodiment a maltodextrin, or in another embodiment milk powder, or in another embodiment a whey protein, or in another embodiment a lipid, or in another embodiment a gum, or in another embodiment a cellulose, or in another embodiment an amorphous lactose, or in another embodiment a combination thereof.

In another embodiment, mixing the prolactin with an appropriate encapsulating material forming a blend further comprises mixing said compound with an encapsulant.

In another embodiment of the invention, the invention provides a method of manufacture of a protected prolactin to retain biological activity of the protein.

The invention may be used to preserve biological activity of a prolactin from adverse temperature, or from adverse pressure, adverse humidity, adverse pH, adverse osmotic concentration, adverse ionic concentration, adverse enzymatic degradation, chemical degradation, presence of metals, surfactants and/or chelators, radiation (including UV, IR, or visible light or combination thereof), microbial degradation or from physical changes including first or second order phase transitions.

In one embodiment, the term “first order phase transition” refers to a discontinuity in the first derivative of Gibbs free energy with temperature at a constant concentration [(<3G/δT)c]. In another embodiment, the term “first order phase transition” refers to crystallization, or in another embodiment, to condensation, or in another embodiment, to evaporation, or in another embodiment, to melting.

In another embodiment, the term “second order phase transition” refers to a discontinuity in the second derivative of Gibbs free energy with temperature at a constant concentration [i.e (95G/δT)c=(9H/δT)c]. In another embodiment, the term “second order phase transition” refers to glass/rubber transition, or in another embodiment, to onset of rotational mobility (δ-transition), or in another embodiment, to onset of vibrational mobility, or in another embodiment, to antemelting.

In another embodiment of the invention, an analogue to the protected prolactin is present in a natural mammalian milk or natural eggs, but its concentration is significantly lower, non viable, non available or non-existent in commercially processed human infant foods or animal infant feeds.

In one embodiment, “Mammal” for purposes of treatment refers to any animal classified as a mammal, including humans, domestic and farm animals, and zoo, sports, or pet animals, such as dogs, horses, cats, hamsters, rats, mice, cattle, pigs, goats, sheep, etc. In another embodiment, the mammal is human.

In another embodiment, concentration as used herein refers to Molar concentration and its fractions, or percentage relative to that existing in colostrum, full milk and eggs.

In one embodiment, the term “significantly lower” refers to the amount of the compound analogue to the prolactin in commercially processed milk being between about 0.01 to about 50 percent of that present in natural unprocessed colostrum, full milk or egg.

In one embodiment, the encapsulating material is food grade, or in another embodiment, the encapsulating material is feed grade, or in another embodiment, the encapsulating material pharmaceutical grade, or in another embodiment is a combination thereto.

In one embodiment, the invention provides a method for preparing at least one encapsulated prolactin in a nutritional food formulation or in a nutritional feed formulation, comprising mixing the prolactin with an appropriate encapsulating material forming a blend, then processing the blend formed to form a functionally multilayered protected dry blend, wherein each of the protective layers is specifically designed in another embodiment, to degrade as a response to change in an environmental trigger and then adding the dry blend to the nutritional food formulation or nutritional feed formulation, wherein the processing of the blend further comprises the forming of a round or non-round core, followed by drying of the core in a fluidized bed dryer, collecting the dehydrated core, suspending the dehydrated blend in a second functional encapsulating liquid, drying the suspension in a fluidized bed dryer and collecting the dehydrated suspension followed by resuspending the suspension obtained in the previous step in a third functional encapsulating fluid, then drying the resuspension a fluidized bed and finally adding the dry blend obtained to the nutritional food formulation or nutritional feed formulation, thereby preparing a multilayered encapsulation of a prolactin in a nutritional food formulation or a nutritional feed formulation. In one embodiment, the initial blend is liquid.

In one embodiment, a second protective layer, or in another embodiment a third protective layer, or in another embodiment a fourth protective layer, or in another embodiment a fifth protective layer, or in another embodiment a sixth protective layer, or in another embodiment a seventh protective layer, or in another embodiment an eighth protective layer, or in another embodiment a ninth protective layer, or in another embodiment a tenth protective layer further comprises a functional encapsulating material such as a maltodextrin, or a vitamin in another embodiment, or an antioxidant in another embodiment, or a protease inhibitor in another embodiment, or a growth hormone in another embodiment, or an EGF (Epidermal Growth Factor) in another embodiment, or an insulin and insulin-like growth factor in another embodiment, or an insulin-like growth factor's binding protein in another embodiment, or an immunoglobulin in another embodiment, or a proline-rich polypeptide in another embodiment, or a lactoferrin in another embodiment, or a protease in another embodiment, or a lactalbumin in another embodiment, or an interleukin in another embodiment, or a lysozyme in another embodiment, a TGFA (Transforming Growth Factor A) in another embodiment, or a PDGF (Platelet Derived Growth Factor) in another embodiment or combination thereof.

In one embodiment, the second, or in another embodiment the third functional encapsulating material or in another embodiment a fourth functional encapsulating material, or in another embodiment a fifth functional encapsulating material, or in another embodiment a sixth functional encapsulating material, or in another embodiment a seventh functional encapsulating material, or in another embodiment an eighth functional encapsulating material, or in another embodiment a ninth functional encapsulating material, or in another embodiment a tenth functional encapsulating material is maltodextrin, which, in another embodiment has a DE value between about 2 to about 64. In one embodiment the maltodextrin has a DE of between about 2 and about 5, or in another embodiment between about 5 and about 10, or in another embodiment between about 10 and about 15, or in another embodiment between about 15 and about 20, or in another embodiment between about 20 and about 25, or in another embodiment between about 25 and about 30, or in another embodiment between about 30 and about 35, or in another embodiment between about 35 and about 40, or in another embodiment between about 45 and about 50, or in another embodiment between about 50 and about 55, or in another embodiment between about 55 and about 60, or in another embodiment between about 60 and about 64. In one embodiment, the maltodextrin has a DE of 18. In another embodiment, the maltodextrin has a DE of 6.

In one embodiment, a protecting layer enables the maintenance of the bioactive properties of the prolactin while in a “dormant state”, which in one embodiment refers to the period when the protected prolactin is dehydrated, such as those present in powdered infant formulas, milk substitute products, and semi-solid/solid mixes and pellets. In another embodiment, the term “dormant state” of the prolactin refers to the preservation of the native tertiary and quaternary structures of the prolactin in an anhydrous state.

In one embodiment of the invention the protecting layer provides protection to the encapsulated prolactin, so that the prolactin shall maintain its bioactive properties in hostile conditions such as high temperatures normally leading in another embodiment to the proteins' denaturation, or in another embodiment, high pressures, or in another embodiment, humidity, or in another embodiment, adverse osmotic pressures, or in another embodiment, high or low pH, or in another embodiment, strong enzymatic degradation, or in another embodiment, high solvent concentration and the like, or in another embodiment, a combination of at least two of the above.

In another embodiment, based on a triggering event, an outer protection layer is dissolved, or in another embodiment outer protection layers are dissolved, and the “dormant” prolactin will be released and become physiologically active.

Preferably, the release of prolactin is essentially while the microcapsules are in contact with different parts of the gastrointestinal tract. In one embodiment, the release is mainly in the small intestine.

In one embodiment, the encapsulated prolactin will be protected from conditions encountered during commercial pelleting and extrusion processes, including but not limited to cold pelleting and extrusion or hot pelleting extrusion, either at standard temperatures and pressures or at conditions different than standard temperatures and pressures.

In another embodiment, the encapsulated prolactin will be protected from conditions encountered during commercial size reduction processes, including processing by colloid mills, both stator rotor of the frusto conical type, as well as crown and tooth type, ball mills, impact mills, jet impingement mills, homogenizing mills, sonication, high velocity mixers or membrane emulsification devices.

In one embodiment, the encapsulated prolactin will be protected from conditions encountered during commercial baking processes, or in another embodiment freezing processes.

In one embodiment, the external functional encapsulating material in the external encapsulating layer is designed to thermally protect the prolactin for no less than 2 minutes at a temperature of no less than 95° C. In another embodiment, the external functional encapsulating material in the external encapsulating layer is designed to thermally protect the prolactin for no less than 1 minute at a temperature of no less than 120° C. In another embodiment, the external functional encapsulating material is designed to protect the prolactin from photolytic enzymes and pH of no more than 4.75. In one embodiment, the external functional encapsulating material is designed to protect the prolactin from any combination of factors as described hereinabove.

In one embodiment, the invention provides a method for supplementing a nutritional food or feed or drink of a mammal, comprising incorporating a nutritional composition for a subject, comprising a prolactin analogous to one found in a natural food source, and a protective layer, wherein release of the prolactin into the subject is in another embodiment the result of an environmental event, in said nutritional food or feed or drink, thereby supplementing said food or feed or drink.

Therefore, according to this aspect of the invention and in one embodiment, a newborn formulation is provided, comprising a prolactin being encapsulated or embedded in a multilayered edible ingredient, which protects and preserves the prolactin making it viable in the newborn.

In one embodiment of the invention, the newborn formulation may be an infant formula or a milk replacement/substitute or semi-solid feed or solid feed for mammal's newborn consumption.

In another embodiment, the term “milk replacer/substitute” refers to any milk replacer/substitute for mammalian neonates wherein the mammals are of the human, bovine, equine, and swine families for examples calf, lamb, pig, cows, sheep, goat, yaez, cats, dogs and horses. In one embodiment, the milk replacer/substitute refers to any milk replacer/substitute, suitable for mammalian neonates, wherein the mammals are of the feline and canine families.

In one embodiment, the plasticized material is carbohydrate polysaccharides, such as in another embodiment, pentosans, or in another embodiment, a physically or chemically modified starch or in another embodiment, cyclodextrin or in other embodiment mixtures thereof.

In another embodiment, the plasticized material is a polymer such as polyvinylpyrrolidone (PVP, Povidone) or other non-hydrophobic polymers such as N-vinylpyrrolidone (NVP) and poly(vinyl)acetate copolymers, (polyvinyl)alcohol chitosan or mixtures thereof. In one embodiment, the plasticized material is cellulose esters, cellulose ethers, and polyethylene glycol. In another embodiment, the plasticized material is a hydrocolloid such as xanthan, carragenan, alginate, gum arabic, gum acacia, gum tragacanth, gum conjac or in another embodiment, a mixtures thereof.

In one embodiment, the plasticized material is glutenins or in another embodiment gliadins, such as in one embodiment, vital wheat gluten or in another embodiment, isolated gluten, or in another embodiment zein, or in another embodiment vegetable or in another embodiment proteins such as protein from soy in one embodiment or milk in another embodiment, or in another embodiment mixtures thereof.

In another embodiment, starches that used in some of the embodiments are physically or chemically modified starches, with amylose/amylopectin ratios of between about 1 to about 0.001, derived from corn, wheat, rice, or potato, tapioca, yuka, arrow root or a combination thereof.

In one embodiment, sources of starch which may be used also include flours from cereals such as corn, wheat, durum wheat, rice, barley, oat, rye or mixtures thereof.

In one embodiment of the present invention, the wall material used is poly (DL-lactide-co-glycolide).

In another embodiment of the invention the food-grade or feed-grade encapsulating material, used in the neonate formulation comprises, polysaccharide, maltodextrin, milk powder, whey protein, lipid, gum, cellulose or combinations thereof.

In one embodiment of the invention the newborn formulation comprises approximately uniformly sized particles of encapsulated plant-extracted prolactin, wherein the particles have a diameter between about 0.1 and about 5,000 micrometers. In one embodiment, D3,2 is the area average particle diameter.

In one embodiment of the present invention, the formulation is used for post weaning mammals. In another embodiment, post- weaning mammals as used herein refer to the age at which the intensively grown mammals are typically weaned off the mother's milk. For example, intensively grown lambs are typically weaned between 25-35 days from birth. Intensively grown piglets are typically weaned between 18-30 days from birth. Intestively-grown calves are typically weaned between 40-70 days from birth.

In all of these newborn animals, in one embodiment of the invention, the provided quantity of the milk replacer containing the prolactin is gradually reduced, and the quantity of the prolactin in mix, pellets or other semi-solid or solid feed is gradually increased.

In another embodiment, the integration of the prolactin in mix/pellets/drink is advantageous for as long as 1-9 months post-birth or in another embodiment post-weaning. Alternatively, the prolactin is beneficial for either 1-2 months, 2-3 months, 3-4 months, 4-5 months, 5-6 months, 6-7 months, 7-8 months, or 8-9 months post-birth or post-weaning.

In one embodiment of the invention, the solid or semi-solid feed formulation may be in the form a mash, or in another embodiment pellets, or in another embodiment granules, or in another embodiment agglomerate, or in another embodiment extrude or in another embodiment combinations thereof.

In another embodiment of the invention, the embedded or encapsulated prolactin maintains or substantially maintains its biological function during the digestion of the food or feed.

In one embodiment of the invention, the embedded or encapsulated prolactin is released upon contact with a liquid.

In another embodiment of the invention, the newborn animal solid or semi-solid feed formulation comprises uniformly sized particles of an encapsulated prolactin, wherein the particles have an average size of between about 10 to about 4000 micrometers.

Products containing protected prolactin according to another embodiment are consumed by a variety of subjects such as preterm infants, post-discharge preterm infants, term infants, babies, toddlers, children, adolescents, adults, elderly humans, or infants or adults of non-human animals, such as bovine, porcine, caprine, feline, canine, equine species or in another embodiment infants or adults of any other non-human animals.

In one embodiment, formulas and milk replacers for preterm infants, specially preterm infants born between weeks 24-36, where such formulas or milk replacers contain a protected or un-protected prolactin, are supplemented with a protected or non-protected bioactive protein prior to consumption, are used to assist in accelerating the development or maturation of the preterm infant's gastrointestinal tract or prevent or reduce the incidence of frequently fatal diseases associated with premature birth, such as NEC (Necrotizing Enterocolitis).

In another embodiment, foods and drinks of preterm or term infants incorporate a protected prolactin or in another embodiment, unprotected prolactin, when provided immediately in one embodiment, or shortly after birth in another embodiment, assist in eliminating or in another embodiment, reducing the onset of autoimmune diseases such as IDDM, Celiac, Inflammatory Bowel Disease, and Crohn's Disease etc.

In one embodiment, a protected prolactin is premixed and packaged in a separate package from the food or feed or drink, or in another embodiment, prior to consumption by a subject, the package containing the protected prolactin is opened, and the protected prolactin is incorporated into the food or feed or drink of a subject, thus creating a bioactive supplemented food or feed or drink of a subject.

In another embodiment a method for encapsulating and embedding prolactin in mammalian newborn formulation is provided, comprising the steps of, (i) mixing the prolactin with an edible food grade or feed grade or pharmaceutical grade encapsulating material forming a liquid blend; (ii) drying of the liquid blend; (iii) coating the dry blend with a additional food grade or feed grade or pharmaceutical grade encapsulating material layer; and (iv) adding the dry blend to the newborn formulation.

In one embodiment the mammalian newborn food formulation may be infant formula or milk replacer/substitute or other drink. Such a formulation is in another embodiment, a form of powder, a solution, a suspension, an emulsion, an ointment, a cream in both liquid, semi-solid or a solid form.

In another embodiment of the invention, a formulation for post weaning mammals which is a solid or a semi-solid formulation is provided, comprising an encapsulated and embedded prolactin prepared by the following process: (i) mixing the compound with a food grade or feed grade or pharmaceutical grade encapsulating material so as to form a liquid blend; (ii) drying of the liquid blend so as to form a dry blend; (iii) coating the dry blend with a additional food grade or feed grade or pharmaceutical grade encapsulating material layer; and (iv) adding the dry blend to the mammalian solid or semi-solid feed formulation. The solid or semi-solid formulation may be in a form of pellets or mash/mix.

Further, according to one embodiment, the step of mixing the prolactin and the wall forming food grade or feed grade or pharmaceutical grade material, involves the addition of liquid, such as, but not limited to: water, saline, alcohol, molasses, organic solvents or similar food grade or feed grade or pharmaceutical grade encapsulating material solvent.

In another embodiment, the ratio between the food grade or feed grade or pharmaceutical grade material and the solvent of the food grade or feed grade or pharmaceutical grade encapsulating material may be in one embodiment between about 1:1 to about 1:1,000, in another between 1:3 and 1:100.

In another embodiment, the dry blend undergoes further size- reduction. The encapsulated prolactin in one embodiment may be further encapsulated by an additional protection layer, which may be formed in another embodiment of the same food grade or feed grade or pharmaceutical grade encapsulating material or, in another embodiment a different food or feed grade or pharmaceutical grade encapsulating material.

In one embodiment, the role of the protective layer is to protect the core from adverse environmental conditions such as temperature, steam and/or pressure, or other environmental triggers. Alternatively or additionally, the protective layer's role is to protect the core from degradation.

In one embodiment, each combination of a different number and type of encapsulation layers result in a unique product suitable for the protection of the prolactin during encapsulation manufacturing conditions, and/or during integration of prolactin into food or feed or drink products and/or during storage, and for maturation of the gastrointestinal system, and/or for properties and characteristics of the subject at the specific age it is being fed. Accordingly, a multi-layer encapsulation for a piglet of 2 days old may substantially differ from multi-layer encapsulation required for a 25 days old piglet.

In one embodiment the dry blend is further mixed with said food or feed grade or pharmaceutical grade encapsulating material so as to form another layer of food grade or feed grade or pharmaceutical grade encapsulating material layer enveloping the prolactin.

In another embodiment of the invention, the food grade or feed grade or pharmaceutical grade encapsulating material is a polysaccharide, milk powder, whey protein, lipid, gum Arabic microcrystalline cellulose, their analogs or combinations thereof.

In one embodiment of the invention the food grade or feed grade or pharmaceutical grade encapsulating material is a solid at temperatures of up to 85° C.

In another embodiment of the invention, the step of drying the food grade or feed grade or pharmaceutical grade encapsulating material and prolactin is done using the methods including but not limited to: freeze drying, vacuum drying, spray drying, osmotic dehydration, fluidized bed dehydration, solvent evaporation dehydration, sonication assisted dehydration, microwave-assisted dehydration, RF-assisted dehydration, either alone or commercially acceptable combinations thereof.

In one embodiment of the invention, the liquid mix is lyophilized after incorporating a prolactin and a food grade or feed grade or pharmaceutical grade encapsulating material ingredient.

In one embodiment lyophilization produces particles containing a protected prolactin and a food grade or feed grade or a pharmaceutical grade encapsulating material in a glassy matrix.

In one embodiment, a flash freezer is employed to dry the liquid mix through the utilization of liquid gas, which is, in one embodiment, nitrogen, or in another embodiment CO₂, or in another embodiment propane, or in another embodiment, any suitable compressible refrigerant gas.

In one embodiment, the size of the droplets will vary between about 10 and about 5,000 micrometers.

In another embodiment the droplets size distribution depends on a variety of parameters such as in one embodiment, freeze sprayer nozzle size, liquid gas temperature, chamber temperature, mix components ratio, mix and gas flow rates, encapsulating material concentration, plasticizer type or freeze chamber wall geometry.

In one embodiment of the invention, the size distribution of the glassy droplets resulting from the process ranges between 50 microns and 1,000 microns.

In one embodiment this treatment results in glassy frozen micro droplets, where each micro droplet contains a protected prolactin, a food grade or feed grade or pharmaceutical grade encapsulating material and the food grade or feed grade or pharmaceutical grade solvent.

In another embodiment once such frozen droplets are placed in temperatures above the melting temperature of the mix, the liquid mix from the previous phase of the process shall be reconstituted.

In one embodiment of the invention, the process further includes the freeze-drying of a combination of prolactin and a food grade or feed grade or pharmaceutical grade encapsulating material.

In another embodiment, freeze drying may be carried out on either a liquid mixture of prolactin and a food grade or feed grade or pharmaceutical grade encapsulating material or on frozen glassy micro droplets as described hereinabove.

In one embodiment the result of this freeze drying process is dry glassy material which includes a food grade or feed grade or pharmaceutical grade encapsulating material and the prolactin.

In another embodiment, freeze drying is performed on a liquid mixture, the result of the process being bulk dry material, porous by nature, containing a glassy matrix of the dried food-grade or feed grade or pharmaceutical grade encapsulating material encapsulating the prolactin.

In one embodiment, freeze-drying is performed on the output of the flash freeze spraying process, resulting in glassy droplets, with the food grade or feed grade or pharmaceutical grade encapsulating material incorporating the prolactin.

In another embodiment, low-temperature spray drying of combination of prolactin and a food grade or feed grade or pharmaceutical grade encapsulating material is carried out.

In one embodiment, the prolactin is dispersed in the food grade or feed grade or pharmaceutical grade encapsulating material and atomized at a maximum temperature of 45° C.

In another embodiment, the maximum temperature is 37° C., preventing denaturation of the prolactin.

In one embodiment, spray drying may be carried out on a liquid mixture of a protected prolactin, a food grade or feed grade or pharmaceutical grade encapsulating material and a chaperon-like protecting protein, resulting in dry material which comprises the food grade or feed grade or pharmaceutical grade encapsulating material and prolactin.

In one embodiment of the invention, the dehydration of the food grade or feed grade or pharmaceutical grade encapsulating material and prolactin is conducted at a temperature, which is preferably below the denaturation temperature of prolactin.

In another embodiment, the dehydration of the food grade or feed grade or pharmaceutical grade encapsulating material and the prolactin is carried out at a temperature below the onset temperature for prolactin's denaturation threshold or degradation threshold.

Preferably, the dehydration of compositions of the encapsulating material and variants of prolactin is carried out at a temperature below the lowest onset temperature of said variant's denaturation threshold or degradation threshold.

In one embodiment of the invention, the dehydration process of the food grade or feed grade or pharmaceutical grade encapsulating material and the prolactin is carried out at a maximum temperature of 50° C.

In another embodiment of the invention, the step of drying the liquid blend results in glassy freeze-dried droplets containing prolactin and a food grade or feed grade or pharmaceutical grade encapsulating material.

In one embodiment of the invention the step of freeze-drying is preceded by a step of spraying the liquid blend through an atomizer in the presence of a liquid gas.

In one embodiment, extrusion is used as an encapsulation method in which a core material is dispersed in a liquid mass of prolactin and a food grade or feed grade or pharmaceutical grade encapsulating material and ultimately formed into microcapsule.

In another embodiment of the invention, encapsulating or embedding protected prolactin in the formulation described above involves an additional step of premixing the blend in a small volume of the newborn formulation or food grade or feed grade or pharmaceutical grade encapsulating material, or semi solid or solid formulation, to ensure homogeneity prior to its mixing with the whole formulation.

In one embodiment of the invention, protection processes suited for use as used herein include, but are not limited to those which produce protected prolactin in the form of a: powder, a micro-encapsulated powder, a nano-encapsulated powder, a liquid, a micro-emulsified liquid, a nano-emulsified liquid, a solution, a micro-emulsified solution, a nano-emulsified solution, a spread, a mash, an ointment, micro droplets, nano-droplets, tablets and solids such as for example, pellets.

In another embodiment of the invention, the encapsulation process includes duplex, W/O/W, 0/W/O, double or multiple emulsions.

In one embodiment of the invention, a mix of prolactin and a food grade or feed grade or pharmaceutical grade encapsulating material and a surfactant selected from the group of surfactants having an HLB value substantially below 7 are suspended in a non-miscible, food grade or feed grade or pharmaceutical grade material and further mixed, affecting size reduction using methods hereinabove mentioned.

In another embodiment, the milled emulsion is further mixed with a food grade or feed grade or pharmaceutical grade material that is miscible with the encapsulating material, and a food grade or feed grade or pharmaceutical grade surfactant selected from the group of surfactants having an HLB value substantially higher than 7 and further reduced in size using one of the methods hereinabove mentioned.

According to an embodiment of the invention, following formulation of a prolactin, micro emulsification or nano emulsification of the formulated prolactin is conducted.

In one embodiment, the formulated prolactin is mixed with an emulsion incorporating water, oil phase and surfactant. As a result of such mixing, the prolactin molecules are reorganized into the dispersed phase of the emulsion.

The protection provided to prolactin by the micro emulsion or nano emulsion in another embodiment, relates to temperature exposure protection, and improved solubility of the prolactin within the food or feed with which it is integrated, following the release of the prolactin from its encapsulation prior to its consumption and/or during the digestion process.

In another embodiment, the prolactin in the nano emulsion or micro emulsion is initially protected within the liquid micro emulsion or liquid nano emulsion.

In one embodiment of the invention, a method is provided for the encapsulation of prolactin in a food grade or feed grade or pharmaceutical grade glassy matrix, the method comprising; (i) mixing a homogeneous intimate mixture between a prolactin and a wall forming, food grade or feed grade or pharmaceutical grade encapsulating material creating a blend, (ii) mixing said blend with an appropriate plasticizer, (iii) rapidly removing said plasticizer while inhibiting crystallization of the wall forming material thereby resulting in encapsulation of the prolactin in a food grade or feed grade or pharmaceutical grade glassy matrix.

In another embodiment of the invention, a method is provided for the encapsulation of a prolactin, comprising; (i) mixing a prolactin with a molten wall-forming food grade or feed grade or pharmaceutical grade encapsulating material, and (ii) rapidly cooling the molten, a wall forming material thereby resulting in encapsulation of the prolactin in a food-grade or feed-grade or pharmaceutical-grade glassy matrix.

“glassy-state matrix” refers to an amorphous metastable solid wherein rapid removal of a plasticizer causes increase in viscosity of the biopolymer to the point where translational mobility of the critical polymer segment length is arrested and alignment corresponding to the polymer's inherent adiabatic expansion coefficient is discontinued.

Hydrophilic materials both of a monomer and a polymeric nature either exist as or can be converted into amorphous states which exhibit the glass/rubber transitions characteristic of amorphous macromolecules. These materials have well defined glass transition temperatures Tg which depend in one embodiment on the molecular weight or in another embodiment on the molecular complexity of the glass forming substance. Tg is depressed by the addition of diluents. Water is the universal plasticizer for all such hydrophilic materials. Therefore, the glass/rubber transition temperature may be adjustable by the addition of water or an aqueous solution, or alternatively by the removal of water or an aqueous solution.

In another embodiment, the plasticizer may be any substance of molecular weight lower than that of the biocompatible polymer that creates an increase in the free interstitial volume. The plasticizer may be an organic compound, which in one embodiment is triglyceride of varying chain length, or in another embodiment water.

In another embodiment of the invention, a method for encapsulating and embedding a prolactin in newborn formulation is provided, the method comprising; (i) mixing prolactin with a liquid food grade or feed grade or pharmaceutical grade encapsulating material so as to form a liquid blend, (ii) drying of the liquid blend so as to form a dry blend; (iii) coating the dry blend with an additional layer comprised of a food grade or feed grade or pharmaceutical grade encapsulating material, where each such layer has different properties relating to environmental conditions in regard of durability and degradation, and (iv) adding the dry blend to the newborn formulation, thereby being a method for encapsulating and embedding a prolactin in newborn formulations.

In one embodiment of the invention, a newborn formulation is provided comprising a prolactin being encapsulated or embedded in a food grade or feed grade or pharmaceutical grade encapsulating material.

In another embodiment of the invention, a method for encapsulating or embedding a prolactin in newborn solid or semi solid feed formulation or newborn drink is provided, comprising the steps of; (i) mixing the prolactin with a liquid food grade or feed grade or pharmaceutical grade encapsulating material so as to form a liquid blend, (ii) drying of the liquid blend so as to form a dry blend, (iii) coating the dry blend with a additional layer comprised of a food grade or feed grade or pharmaceutical grade encapsulating material, where each such layer has different durability and degradation properties relating to environmental conditions, and (iv) adding the dry blend to the newborn animal solid or semi solid feed formulation of newborn drink, thereby being a method for encapsulating and embedding the prolactin in newborn animal solid or semi solid feed formulation or newborn drink.

In another embodiment of the invention, a newborn animal solid or semi-solid feed formulation or newborn drink is provided, comprising a prolactin being encapsulated or embedded in a food grade or feed grade or pharmaceutical grade material.

The following examples are presented in order to more fully illustrate some embodiment of the invention. They should, in no way be construed, however, as limiting the scope of the invention.

### EXAMPLE 1

### Fluidized Bed Coating Process Specifications

### Coating Samples

PMDP—Polycose® (core)+[MD (maltodextrin)+prolactin] (coating solution)−→(one concentration−2 IU/gr)

[LMDP—Lactose (core)+[MD+prolactin] (coating solution)−>(one concentration−2 IU/gr)

MMDP—Maltodextrin (core)+[MD+prolactin] (coating solution)−>(one concentration−2 IU/gr; MD 18 concentrations of 10%, 20%, 30% MD 18+Vitamin C 10% (one concentration on each core and MD 18 coating)

### Coating Conditions

The mixing is done under food grade regulation conditions and with compliance with the Biodar ISO9001:2000 quality system procedures. Throughout the manufacturing process the product temperature does not exceed 37° C. The process is performed at a slow rate to prevent agglomeration.

### Sampling

From each stage in the process a sample of 10 grams is taken, packed in a bag and labeled to indicate the sample number.

### EXAMPLE 2

### Prolactin Solution Premix Preparation

### Mixing Conditions

Mixing is done under cGMP conditions and with compliance with HACCP procedures

### Solution Preparation

A Maltodextrin DE-18, prolactin and saline 0.45% solution is prepared using 20% Maltodextrin DE-18, prolactin (100 IU/ml) at a ratio of 100 cc to 500 gr active ingredient coated core (MD/Polycose core coated with MD+prolactin layer). Saline 0.45% is added to complete to a 100% solution. Saline is added partially to the solution. The rest of the Saline solution is used to rinse the prolactin bottles to ensure all material has been washed out and added to the solution. [The solution is mixed until the Maltodextrin is completely dissolved.

### EXAMPLE 3

### In Vitro Testing

Several in-vitro tests are performed on the prolactin product in order to verify that the manufacturing process does not adversely affect the required product characteristics and bioactivity, and further to ensure that it consistently meets its technical specifications.

### Osmolarity Testing

The Osmolarity testing should indicate that the addition of prolactin microcapsule powder to the RTF formula has no appreciable effect on the final solution osmolarity, thus the formula remaining within its specifications. The RTF (Ready-To-Feed) liquid formula has a defined osmolarity that is important for suitable nutrients consumption. Therefore, a test is performed to verify that the addition of prolactin microcapsule powder to the RTF does not change the osmolarity of the liquid formula. The test is performed by immersing 1.0 g to 1.5 g of prolactin microcapsule powder in 60 ml preterm RTF formula bottle, analyzing the osmolarity and comparing it to a control containing the same RTF formula without prolactin. Each sample is analyzed in triplicates.

### Product Stability in Liquid Infant Formula Over Time

The prolactin microcapsule powder is intended to be consumed immediately after solubilization in the infant formula. Nevertheless, the prolactin stability over time is measured by adding a pre-defined quantity of liquid prolactin (concentration of 100 μU per aliquot) to 60 ml Materna™ preterm infants formula in a baby bottle, and the prolactin quantity is analyzed immediately following addition, then after 3, 6, 9, 12, 15, 18, 21 and 24 hours by using the Elisa kit. Furthermore, in order to evaluate the final product homogeneity, at each time interval, sampling is taken at the upper, middle and lower layers of the sampled RTF bottle, as well as after formula liquid stirring.

### Prolactin Microcapsule Powder Stability at Extreme Temperatures

Since prolactin is a temperature sensitive protein, the product ability to protect the prolactin component following exposure of the encapsulated powder to high temperature for various durations, is measured.

A person holding ordinary skill in the art would readily recognize that this invention is not limited in its application to the details of construction and the arrangement of components set hereinabove in the mentioned description. It should be appreciated that various modifications can be made without materially changing the scope or spirit of the current invention. It should be noted that practicing the invention is not limited to the applications hereinabove mentioned and many other applications and alterations may be made without departing from the intended scope of the present invention. Also, it is to be understood that the lexicography employed herein is for the purpose of description and should not be taken as limiting.

It should be clear that the description of the embodiments and attached Figures set forth in this specification serves only for a better understanding of the invention, without limiting its scope as covered by the following Claims.

It should also be clear that a person skilled in the art, after reading the present specification, can make adjustments or amendments to the attached Figures and above described embodiments that would still be covered by the following Claims.

1. A method of enhancing small intestinal growth, small intestinal functional maturation and/or calcium absorption across intestinal epithelial membranes enhancing gut maturation of a subject, the method comprising: providing the subject with a nutritional composition, the composition comprising prolactin and at least one protective layer, and releasing said prolactin from the composition in said subject as a result of an environmental change, thereby enhancing the small intestinal growth, small intestinal functional maturation and/or calcium absorption across intestinal epithelial membranes enhancing gut maturation, wherein said subject is a human selected from the group consisting of a neonate, a preterm human infant, a term human infant, a baby, a toddler, an adolescent, an adult and an old person.

2. The method of claim 2, wherein the prolactin is identical or similar or analogous to the prolactin found in a natural food source selected from the group consisting of natural unprocessed milk, natural unprocessed eggs, animal tissue, colostrum and serum.

3. The method of claim 2, wherein the prolactin in obtained by at least one of extracting prolactin from milk, eggs, animal tissue, serum or colostrums; harvesting prolactin from recombinant DNA technology; and synthetically producing prolactin.

4. The method of claim 2, wherein the composition is specifically formulated for said subject.

5. The method of claim 2, wherein the prolactin is selected from the group consisting of non-glycosylated prolactin, non-phosphorylated prolactin, a prolactin monomer and any combination thereof.

6. The method of claim 2, wherein the composition comprises at least one complex between the prolactin and the at least one protective layer.

7. The method of claim 2, wherein the composition further comprises one or more water-soluble polymers selected from the group consisting of polyethylene glycol, copolymers of polyethylene glycol and polypropylene glycol, carboxymethyl cellulose, dextran, polyvinyl alcohol, polyvinylpyrrolidone and polyproline, for complexation between the prolactin and the at least one protective layer via covalent attachment of the water-soluble polymers to both the prolactin and the protective layer.

8. The method of claim 2, wherein the at least one protective layer is specifically designed to degrade as a response to specific environmental change in at least one of temperature, moisture content, pressure, pH, ionic strength, enzymatic activity, time passed since providing the subject with the nutritional composition, and any combination thereof

9. The method of claim 8, wherein the at least one protective layer is selected from the group comprising polysaccharide, maltodextrin, milk powder, whey protein, lipid, gum, celluloses, amorphous lactose and combinations thereof

11. A method of enhancing small intestinal growth, small intestinal functional maturation and/or calcium absorption across intestinal epithelial membranes enhancing gut maturation of a subject, the method comprising: providing the subject with a supplemented nutritional food or feed or drink comprising nutritional food and a prolactin composition, the prolactin composition comprising prolactin at least one protective layer, wherein release of said prolactin from the composition in said subject is the result of an environmental change thereby enhancing the small intestinal growth, small intestinal functional maturation and/or calcium absorption across intestinal epithelial membranes enhancing gut maturation, wherein said subject is a human selected from the group consisting of a neonate, a preterm human infant, a term human infant, a baby, a toddler, an adolescent, an adult and an old person.

12. The method of claim 11, wherein the supplemented nutritional food or feed or drink is produced by adding the prolactin composition to said nutritional food or feed or drink.

13. The method of claim 11, wherein the supplemented nutritional food or feed or drink is produced by a method comprising:
a. preparing a protected prolactin comprising: mixing the prolactin with an appropriate first protective material forming a first blend;
b. processing the first blend to form a dry second blend comprising prolactin in at least one protective layer, wherein said protective layer is specifically designed to degrade as a response to change in an environmental trigger; and
c. adding the dry second blend to said nutritional food or nutritional feed or drink.

14. The method of claim 13, wherein preparing a protected prolactin further comprises:
d. forming a round core from the second blend;
e. drying the core;
f. collecting the dehydrated core;
g. making a first suspension comprising the dehydrated core in a liquid second protective material;
h. drying the first suspension in a fluidized bed drier;
i. collecting the fluidized bed-dried suspension;
j. making a second suspension comprising the collected dried suspension in a liquid third protective material; and
k. drying the second suspension in a fluidized bed drier.

15. The method of claim 13, wherein the first blend is liquid.

16. The method of claim 14, wherein the first suspension further comprises at least one of a maltodextrin, a vitamin, an antioxidant, a protease inhibitor, a growth hormone, an EGF (Epidermal Growth Factor), an insulin and insulin-like growth factor, an insulin-like growth factor's binding protein, an immunoglobulin, a proline-rich polypeptide, a lactoferrin, a protease, a lactalbumin, an interleukin, a lysozyme, a TGFA (Transforming Growth Factor A), a PDGF (Platelet Derived Growth Factor) or combination thereof.

17. The method of claim 14, wherein the second suspension further comprises at least one of a maltodextrin, a vitamin, an antioxidant, a protease inhibitor, a growth hormone, an EGF (Epidermal Growth Factor), an insulin and insulin-like growth factor, an insulin-like growth factor's binding protein, an immunoglobulins, a proline-rich polypeptide, a lactoferrin, a protease, a lactalbumin, an interleukin, a lysozyme, a TGFA (Transforming Growth Factor A), a PDGF (Platelet Derived Growth Factor) or combination thereof.

18. The method of claim 13, further comprising an agglomeration step.

19. The method of claim 18, wherein the agglomeration step results in particle average diameter between about 0.1 and about 5,000 micrometers.
